(19) United States
(12) Patent Application Publication
(10) Pub. No.: US 2020/0075129 A1
(43) Pub. Date: Mar. 5, 2020

(54) MACHINE LEARNING SYSTEM FOR GENOTYPING PCR ASSAYS

(71) Applicant: LIFE TECHNOLOGIES CORPORATION, Carlsbad, CA (US)

(72) Inventors: Daqing WANG, San Mateo, CA (US); Pius BRZOSKA, Woodside, CA (US); Elliot SHELTON, San Mateo, CA (US)

(21) Appl. No.: 16/553,993

(22) Filed: Aug. 28, 2019

Related U.S. Application Data
(60) Provisional application No. 62/725,171, filed on Aug. 30, 2018.

(51) Int. Cl. G16B 40/20; G06N 20/10; C12Q 1/686; G16B 20/00

(52) U.S. Cl. CPC G16B 40/20; G16B 20/00; C12Q 1/686; G06N 20/10

(57) ABSTRACT

A quality control system for a qPCR receives signals resulting from operation of the qPCR system on an assay, and applies labeled data sets to a Support Vector Machine (SVM) to generate classifications for the signals to generate classifications that are utilized as operational feedback to the qPCR system.

### CROSS-REFERENCE TO RELATED APPLICATIONS

This application claims the benefit of priority to U.S. Provisional Patent Application No. 62/725,171, filed Aug. 30, 2018, which disclosure is herein incorporated by reference in its entirety.

### BACKGROUND

Some conventional PCR assay genotyping methodologies (e.g., Taqman®) are based on unsupervised centroid Minimum Cluster Separation Sigma (MCSS) algorithms. A MCSS cutoff (5.0 for example) is empirically selected to tag assays as failure or pass during quality control (QC). However, the hard cutoff means that assays are not classified with nuance. For example, if the cutoff is 5.0, MCSS=5.0 results in a QC pass classification, while MCSS=4.9 results in a QC failure classification. This leads to QC failure of many products that might be acceptable, and thus increases manufacturing loss.

### SUMMARY

A new classification methodology for assay arrays is disclosed based on Support Vector Machine classification and learning, and which may be implemented to genotype cell lines and biological samples. The new methodology improves the problematic ambiguity of prior QC methods by factoring in historical genotyping results through model training to classify genotypes and to tag qPCR reactions and samples with genotype classifications.

### BRIEF DESCRIPTION OF THE DRAWINGS

To easily identify the discussion of any particular element or act, the most significant digit or digits in a reference number refer to the figure number in which that element is first introduced.

FIG. 1 illustrates a process 100 in accordance with one embodiment.

FIG. 2 illustrates a qPCR system 200 in accordance with one embodiment.

FIG. 3 illustrates a plate preparation 300 in accordance with one embodiment.

FIG. 4 illustrates a genotyping system 400 in accordance with one embodiment.

FIG. 5 illustrates a radial algorithm 500 in accordance with one embodiment.

FIG. 6 illustrates an SVM qPCR assay model 600 in accordance with one embodiment.

FIG. 7 illustrates a cloud learning and control system 700 in accordance with one embodiment.

FIG. 8 is an example block diagram of a computing device 800 that may incorporate embodiments of the present invention.

### DETAILED DESCRIPTION

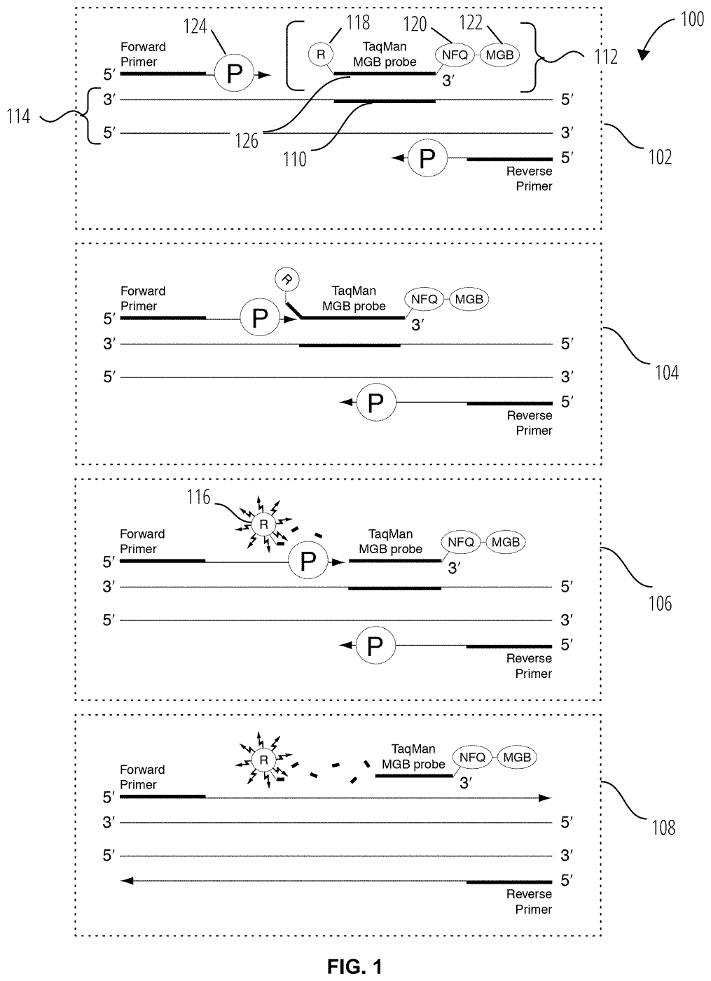

FIG. 1 illustrates a process 100 in accordance with one embodiment utilized in PCR amplification, specifically a 5′ nuclease assay utilized in PCR amplification. The process 100 shows four stages of this assay process that occur in every cycle and which do not interfere with the exponential accumulation of product. The four stages include a polymerization phase 102, a strand displacement phase 104, a cleavage phase 106, and a completion phase 108. During the polymerization phase 102 a forward primer and a reverse primer start replicating a section of a double stranded DNA 114 near a target sequence 110. The forwards primer (5′→3′) comprises a hot-start polymerase 124 (Taq polymerase), that functions at temperatures that DNA polymerase is inactive avoiding unwanted replication. A probe comprises a reporter dye 118, a complementary sequence 126, a non-fluorescent quencher 120, and a minor groove binder 122. The probe hybridizes to a target sequence 110 through the complementary sequence 126. The non-fluorescent quencher 120 and the minor groove binder 122 function as molecules are attached to the 3′ end of probe. When the probe is intact, the non-fluorescent quencher 120 (NFQ) prevents the reporter dye 118 from emitting a fluorescence signal. Because the non-fluorescent quencher 120 does not fluoresce, it produces lower background signals, resulting in improved precision in quantification. The minor groove binder 122 (MGB) increases the melting temperature (Tm) of the probe without increasing its length, allowing for the design of shorter probes. During the polymerization phase 102, the hot-start polymerase 124 moves towards the probe with the reporter dye 118 attached to the 5′ side of the probe.

In the strand displacement phase 104 the hot-start polymerase 124 interacts with the hybridized probe displacing the reporter dye 118. In cleavage phase 106, the hot-start polymerase 124 cleaves the reporter dye 118 from the probe. Cleavage separates the reporter dye from the quencher dye; with the non-fluorescent quencher 120 no longer block the reporter dye 118, the separated reporter dye 116 increases its fluorescence. The increase in fluorescence occurs only if the target sequence is complementary to the probe and is amplified during PCR. The instrument detects the fluorescence from the reporter dye indicating the presence of the target sequence on the double stranded DNA 114. Due to the hybridization of the probe to the target sequence 110, the hot-start polymerase 124 stops at the complementary sequence 126 indicating the completion phase 108.

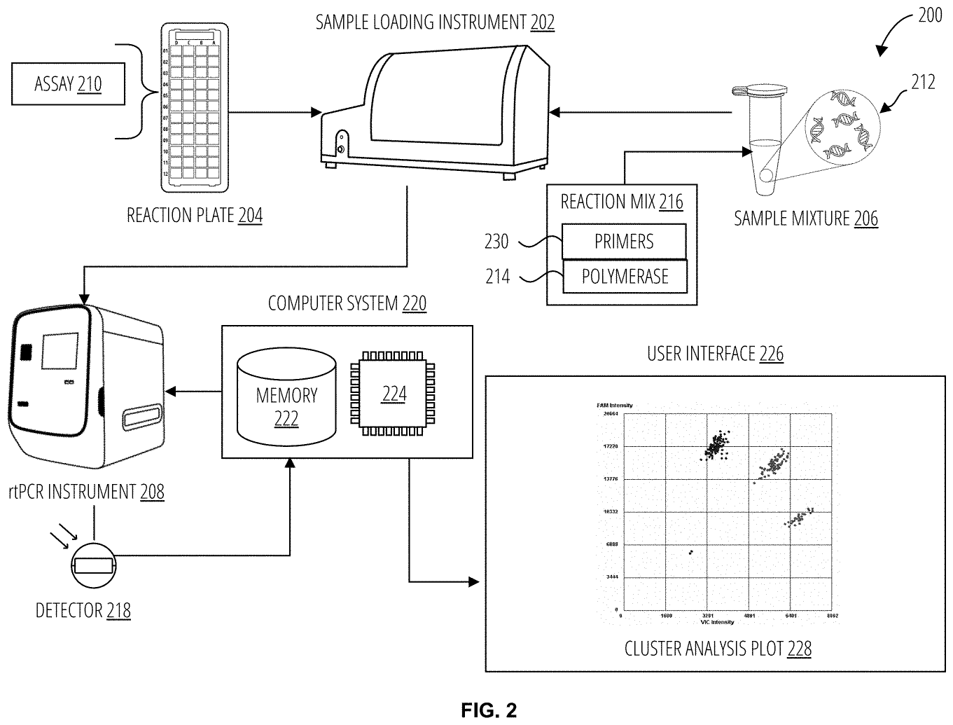

FIG. 2 illustrates a qPCR system 200 comprising a reaction plate 204, a sample loading instrument 202, a real-time PCR instrument 208, a sample mixture 206, a computer system 220, and a user interface 226. The reaction plate 204 comprises a plurality of subarrays each comprising a plurality of through holes that serve as the reaction location for a qPCR experiment. Each of the through-holes may be coated with an assay 210. In some configurations, the assay 210 is a probe that specifically targets a nucleotide sequence in the sample DNA. During the amplification of the sample DNA, the probe indicates its presence of the target sequence through the release of a reporter dye that is detected by the real-time PCR instrument 208. The reaction plate 204 is combined with the target polynucleotide sequences 212 in a sample loading instrument 202. Prior to combining the reaction plate 204 with the target polynucleotide sequences 212, the target polynucleotide sequences 212 are prepared in a sample mixture 206 comprising a reaction mix 216. The reaction mix 216 comprises, at least, polymerase 214 and primers 230. The polymerase 214 amplifies the doubled stranded DNA during the PCR reaction. The sample loading instrument 202 loads a specific volume of the sample mixture 206 into each intended through-hole in the reaction plate 204. When the sample loading instrument 202 has completed its preparation of the reaction plate 204, the reaction plate 204 is loaded into the real-time PCR instrument 208. The real-time PCR instrument 208 is configured by the computer system 220 to operate thermocycler that cycles through different temperatures ranges that triggers specific phases for DNA replication. When the reaction plate 204 it undergoes several cycles of replication that includes a high heat phase (94-98° C. (201-208° F.)) that denatures the DNA strand breaking the hydrogen bonds between complementary bases, yielding two single-stranded DNA molecules. The denaturing phase is followed by the annealing phase where the reaction temperature is lowered to 50-65° C. (122-149° F.) for 20-40 seconds. The annealing phase allows for the annealing of primers and the probe sets to the target sequences in the DNA. The annealing phase is followed by an extension/elongation phase where the temperature is adjusted to the optimum activity temperature for the thermostable DNA polymerase of Taq (Thermus aquaticus) polymerase which is approximately 75-80° C. (167-176° F.). During the elongation/extension phase the polymerase synthesizes a complementary strand starting next to the location of a primer and continues until synthesizing the new complementary strand until it abuts with a probe on the target sequence. When the polymerase interacts with the probe, the probe releases a fluorescent marker that is detected by the detector 218 of the real-time PCR instrument 208. Information from the detector 218 is recorded by the computer system 220 as a first signal that corresponds to one of the through holes. The detected signals are reported to a computer system 220 comprising a memory 222 and a processor 224 that stores and processes the information to generate a cluster analysis plot 228 showing the copy number and instances of the target sequences in the sample mixture 206. The computer system 220 communicates genotyping results to the user interface 226 to display the cluster analysis plot 228.

As one of ordinary skill in the art is apprised, a PCR analysis is performed on a thermal cycling instrument, which has various protocols for cycling though a plurality of thermal cycles in order to amplify a gene target. In various embodiments of the present teachings, the number of cycles performed for the amplification may be between about 20-40 cycles. For various embodiments of the present teachings, the number of cycles performed for the amplification may be greater than 40 cycles. For amplification of a gene target a thermal cycling instrument may perform a first thermal cycle of a PCR experiment in a certain cycle time that may be associated with a first thermal cycle number.

In various embodiments of a genotyping analysis, two or more DNA samples are probed with a first probe and a second probe. A processor may receive from a qPCR instrument based on any of a variety of protocols for data collection, a first data set at a first time that includes for each of the two or more DNA samples a first probe intensity and a second probe intensity at the first time. A processor may receive from a qPCR instrument based on any of a variety of protocols for data collection, a second data set at a second time that includes for each of the two or more DNA samples a first probe intensity and a second probe intensity at the second time.

According to various embodiments of the present teachings, a user interface may present to an end user a visualization tool for the analysis of the data sets received a first time and a second time. As previously mentioned, a plurality of samples may be processed for genotyping analysis in a batch, yielding data-intense data sets. Various embodiments of a systems and methods according to the present teachings provide for embodiments of a visualization tool that may assist an end user in the evaluation and analysis of such data-intense data sets. For various embodiments of systems and methods according to the present teachings, in response to input from an end user, a processor may generate a first plot of first probe intensity versus a second probe intensity using the first data set. Further, a processor may generate a second plot of first probe intensity as a function of second probe intensity using the second data set in response to input from an end user. According to various embodiments of systems and methods of the present teachings, a processor may display the first plot and the second plot in response to input from an end user. In various embodiments, the input may be an interactive process with a user interface to display the data in a step-wise fashion. In such embodiments, an end user may select any data set in any order for display.

In various embodiments, a processor may receive data during the run time of a PCR experiment. For example, a processor may receive the first data set from a qPCR instrument after the collection of the first data set and before collection of the second data set. Further, this protocol may be extended throughout the run time, so that, for example, a processor may receive the second data set from a qPCR instrument after the collection of the second data set and before collection of a subsequent data set.

In some embodiments, a processor may receive the first data set and the second data set from a qPCR instrument after thermal cycling has completed. For example, a processor may receive the first data set and the second data set after it has been stored on a computer-readable medium.

In some configurations, a visualization tool may assist an end user in the displaying of various aspects of genotyping data sets, thereby facilitating in the analysis of genotyping data. In various embodiments, a processor may display a plot showing trajectory lines between the second data set and the first data set. In various embodiments, a processor may display on the first plot quality values for the first data set and displays on the second plot quality values for the second data set. According to various embodiments, a user interface provides an interaction between selections made on a sample table and dynamically displayed on a plot of genotyping data. In various embodiments, selections made by an end user from a user interface of a visualization tool may, for example, but not limited by, provide dynamic analysis for enabling an end user to, for example, but not limited by, troubleshoot ambiguous end-point data, make manual calls, use trajectory lines to assist in visualizing clusters to enhance genotype assignment, optimize assay conditions (i.e. labeling probe, assay buffer, etc.) and optimize analysis conditions.

Various embodiments, the system utilize data sets that may be represented, for example, but not limited by, according to the graph depicted in the cluster analysis plot 228. Such a representation may arise from analyses utilizing two dyes having emissions at different wavelengths, which dyes can be associated with each of a labeling probe directed at one of two alleles for a genomic locus in a biological sample. In such duplex reactions, a discrete set of signals for each of three possible genotypes is produced. In a Cartesian coordinate system of signal 2 versus signal 1, as shown in the cluster analysis plot, each data point shown on such a graphic representation may have coordinates in one of three discrete sets of signals given. Accordingly, for each data point, a discrete set of signals for a plurality of samples may be stored as data points in a data set. Such data sets may be stored in a variety of computer readable media, and analyzed either dynamically during analysis or post analysis, as will be discussed in more detail subsequently.

One such type of assay used to demonstrate the features of embodiments of methods and systems for the visualization of genotyping data can utilize TaqMan® reagents, and may use, for example, but not limited by, FAM and VIC dye labels, as will be discussed subsequently. However, one of ordinary skill in the art will recognize that a variety of assays including labeling probe reagents may be utilized to produce data that may be analyzed according to various embodiments of methods and systems of the present teachings.

The term “labeling probe” generally, according to various embodiments, refers to a molecule used in an amplification reaction, typically for quantitative or qPCR analysis, as well as end-point analysis. Such labeling probes may be used to monitor the amplification of the target polynucleotide. In some embodiments, oligonucleotide labeling probes present in an amplification reaction are suitable for monitoring the amount of amplicon(s) produced as a function of time. Such oligonucleotide labeling probes include, but are not limited to, the 5′-exonuclease assay TaqMan® labeling probes described herein (see also U.S. Pat. No. 5,538,848), various stem-loop molecular beacons (see e.g., U.S. Pat. Nos. 6,103,476 and 5,925,517 and Tyagi and Kramer, 1996, Nature Biotechnology 14:303-308), stemless or linear beacons (see, e.g., WO 99/21881), PNA Molecular Beacons™ (see, e.g., U.S. Pat. Nos. 6,355,421 and 6,593,091), linear PNA beacons (see, e.g., Kubista et al., 2001, SPIE 4264:53-58), non-FRET labeling probes (see, e.g., U.S. Pat. No. 6,150,097), Sunrise®/Amplifluor® labeling probes (U.S. Pat. No. 6,548,250), stem-loop and duplex Scorpion™ labeling probes (Solinas et al., 2001, Nucleic Acids Research 29:E96 and U.S. Pat. No. 6,589,743), bulge loop labeling probes (U.S. Pat. No. 6,590,091), pseudo knot labeling probes (U.S. Pat. No. 6,589,250), cyclicons (U.S. Pat. No. 6,383,752), MGB Eclipse™ probe (Epoch Biosciences), hairpin labeling probes (U.S. Pat. No. 6,596,490), peptide nucleic acid (PNA) light-up labeling probes, self-assembled nanoparticle labeling probes, and ferrocene-modified labeling probes described, for example, in U.S. Pat. No. 6,485,901; Mhlanga et al., 2001, Methods 25:463-471; Whitcombe et al., 1999, Nature Biotechnology. 17:804-807; Isacsson et al., 2000, Molecular Cell Labeling probes. 14:321-328; Svanvik et al., 2000, Anal Biochem. 281:26-35; Wolffs et al., 2001, Biotechniques 766:769-771; Tsourkas et al., 2002, Nucleic Acids Research. 30:4208-4215; Riccelli et al., 2002, Nucleic Acids Research 30:4088-4093; Zhang et al., 2002 Shanghai. 34:329-332; Maxwell et al., 2002, J. Am. Chem. Soc. 124:9606-9612; Broude et al., 2002, Trends Biotechnol. 20:249-56; Huang et al., 2002, Chem Res. Toxicol. 15:118-126; and Yu et al., 2001, J. Am. Chem. Soc 14:11155-11161. Labeling probes can also comprise black hole quenchers (Biosearch), Iowa Black (IDT), QSY quencher (Molecular Labeling probes), and Dabsyl and Dabcel sulfonate/carboxylate Quenchers (Epoch). Labeling probes can also comprise two labeling probes, wherein for example a fluorophore is on one probe, and a quencher on the other, wherein hybridization of the two labeling probes together on a target quenches the signal, or wherein hybridization on target alters the signal signature via a change in fluorescence. Labeling probes can also comprise sulfonate derivatives of fluorescenin dyes with a sulfonic acid group instead of the carboxylate group, phosphoramidite forms of fluorescein, phosphoramidite forms of CY 5 (available for example from Amersham).

As used herein, the term “nucleic acid sample” refers to nucleic acid found in biological samples according to the present teachings. It is contemplated that samples may be collected invasively or noninvasively. The sample can be on, in, within, from or found in conjunction with a fiber, fabric, cigarette, chewing gum, adhesive material, soil or inanimate objects. “Sample” as used herein, is used in its broadest sense and refers to a sample containing a nucleic acid from which a gene target or target polynucleotide may be derived. A sample can comprise a cell, chromosomes isolated from a cell (e.g., a spread of metaphase chromosomes), genomic DNA, RNA, cDNA and the like. Samples can be of animal or vegetable origins encompassing any organism containing nucleic acid, including, but not limited to, plants, livestock, household pets, and human samples, and can be derived from a plurality of sources. These sources may include, but are not limited to, whole blood, hair, blood, urine, tissue biopsy, lymph, bone, bone marrow, tooth, amniotic fluid, hair, skin, semen, anal secretions, vaginal secretions, perspiration, saliva, buccal swabs, various environmental samples (for example, agricultural, water, and soil), research samples, purified samples, and lysed cells. It will be appreciated that nucleic acid samples containing target polynucleotide sequences can be isolated from samples using any of a variety of sample preparation procedures known in the art, for example, including the use of such procedures as mechanical force, sonication, restriction endonuclease cleavage, or any method known in the art.

The terms “target polynucleotide,” “gene target” and the like as used herein are used interchangeably herein and refer to a particular nucleic acid sequence of interest. The “target” can be a polynucleotide sequence that is sought to be amplified and can exist in the presence of other nucleic acid molecules or within a larger nucleic acid molecule. The target polynucleotide can be obtained from any source, and can comprise any number of different compositional components. For example, the target can be nucleic acid (e.g. DNA or RNA). The target can be methylated, non-methylated, or both. Further, it will be appreciated that “target” used in the context of a particular nucleic acid sequence of interest additionally refers to surrogates thereof, for example amplification products, and native sequences. In some embodiments, a particular nucleic acid sequence of interest is a short DNA molecule derived from a degraded source, such as can be found in, for example, but not limited to, forensics samples. A particular nucleic acid sequence of interest of the present teachings can be derived from any of a number of organisms and sources, as recited above.

As used herein, “DNA” refers to deoxyribonucleic acid in its various forms as understood in the art, such as genomic DNA, cDNA, isolated nucleic acid molecules, vector DNA, and chromosomal DNA. “Nucleic acid” refers to DNA or RNA in any form. Examples of isolated nucleic acid molecules include, but are not limited to, recombinant DNA molecules contained in a vector, recombinant DNA molecules maintained in a heterologous host cell, partially or substantially purified nucleic acid molecules, and synthetic DNA molecules. Typically, an “isolated” nucleic acid is free of sequences which naturally flank the nucleic acid (i.e., sequences located at the 5′ and 3′ ends of the nucleic acid) in the genomic DNA of the organism from which the nucleic acid is derived. Moreover, an “isolated” nucleic acid molecule, such as a cDNA molecule, is generally substantially free of other cellular material or culture medium when produced by recombinant techniques, or free of chemical precursors or other chemicals when chemically synthesized.

In some embodiments, PCR amplification products may be detected by fluorescent dyes conjugated to the PCR amplification primers, for example as described in PCT patent application WO 2009/059049. PCR amplification products can also be detected by other techniques, including, but not limited to, the staining of amplification products, e.g. silver staining and the like.

In some embodiments, detecting comprises an instrument, i.e., using an automated or semi-automated detecting means that can, but needs not, comprise a computer algorithm. In some embodiments, the instrument is portable, transportable or comprises a portable component which can be inserted into a less mobile or transportable component, e.g., residing in a laboratory, hospital or other environment in which detection of amplification products is conducted. In certain embodiments, the detecting step is combined with or is a continuation of at least one amplification step, one sequencing step, one isolation step, one separating step, for example but not limited to a capillary electrophoresis instrument comprising at least one fluorescent scanner and at least one graphing, recording, or readout component; a chromatography column coupled with an absorbance monitor or fluorescence scanner and a graph recorder; a chromatography column coupled with a mass spectrometer comprising a recording and/or a detection component; a spectrophotometer instrument comprising at least one UV/visible light scanner and at least one graphing, recording, or readout component; a microarray with a data recording device such as a scanner or CCD camera; or a sequencing instrument with detection components selected from a sequencing instrument comprising at least one fluorescent scanner and at least one graphing, recording, or readout component, a sequencing by synthesis instrument comprising fluorophore-labeled, reversible-terminator nucleotides, a pyro sequencing method comprising detection of pyrophosphate (PPi) release following incorporation of a nucleotide by DNA polymerase, pair-end sequencing, polony sequencing, single molecule sequencing, nanopore sequencing, and sequencing by hybridization or by ligation as discussed in Lin, B. et al. “Recent Patents on Biomedical Engineering (2008)1(1)60-67, incorporated by reference herein.

In certain embodiments, the detecting step is combined with an amplifying step, for example but not limited to, real-time analysis such as Q-PCR. Exemplary means for performing a detecting step include the ABI PRISM® Genetic Analyzer instrument series, the ABI PRISM® DNA Analyzer instrument series, the ABI PRISM® Sequence Detection Systems instrument series, and the Applied Biosystems Real-Time PCR instrument series (all from Applied Biosystems); and microarrays and related software such as the Applied Biosystems microarray and Applied Biosystems 1700 Chemiluminescent Microarray Analyzer and other commercially available microarray and analysis systems available from Affymetrix, Agilent, and Amersham Biosciences, among others (see also Gerry et al., J. Mol. Biol. 292:251-62, 1999; De Bellis et al., Minerva Biotec 14:247-52, 2002; and Stears et al., Nat. Med. 9:140-45, including supplements, 2003) or bead array platforms (Illumina, San Diego, Calif.). Exemplary software includes GeneMapper™ Software, GeneScan® Analysis Software, and Genotyper® software (all from Applied Biosystems).

In some embodiments, an amplification product can be detected and quantified based on the mass-to-charge ratio of at least a part of the amplicon (m/z). For example, in some embodiments, a primer comprises a mass spectrometry-compatible reporter group, including without limitation, mass tags, charge tags, cleavable portions, or isotopes that are incorporated into an amplification product and can be used for mass spectrometer detection (see, e.g., Haff and Smirnov, Nucl. Acids Res. 25:3749-50, 1997; and Sauer et al., Nucl. Acids Res. 31:e63, 2003). An amplification product can be detected by mass spectrometry. In some embodiments, a primer comprises a restriction enzyme site, a cleavable portion, or the like, to facilitate release of a part of an amplification product for detection. In certain embodiments, a multiplicity of amplification products are separated by liquid chromatography or capillary electrophoresis, subjected to ESI or to MALDI, and detected by mass spectrometry. Descriptions of mass spectrometry can be found in, among other places, The Expanding Role of Mass Spectrometry in Biotechnology, Gary Siuzdak, MCC Press, 2003.

In some embodiments, detecting comprises a manual or visual readout or evaluation, or combinations thereof. In some embodiments, detecting comprises an automated or semi-automated digital or analog readout. In some embodiments, detecting comprises real-time or endpoint analysis. In some embodiments, detecting comprises a microfluidic device, including without limitation, a TaqMan® Low Density Array (Applied Biosystems). In some embodiments, detecting comprises a real-time detection instrument. Exemplary real-time instruments include, the ABI PRISM® 7000 Sequence Detection System, the ABI PRISM® 7700 Sequence Detection System, the Applied Biosystems 7300 Real-Time PCR System, the Applied Biosystems 7500 Real-Time PCR System, the Applied Biosystems 7900 HT Fast Real-Time PCR System (all from Applied Biosystems); the LightCycler™ System (Roche Molecular); the Mx3000P™ Real-Time PCR System, the Mx3005P™ Real-Time PCR System, and the Mx4000® Multiplex Quantitative PCR System (Stratagene, La Jolla, Calif.); and the Smart Cycler System (Cepheid, distributed by Fisher Scientific). Descriptions of real-time instruments can be found in, among other places, their respective manufacturer's user's manuals; McPherson; DNA Amplification: Current Technologies and Applications, Demidov and Broude, eds., Horizon Bioscience, 2004; and U.S. Pat. No. 6,814,934.

The term “amplification reaction mixture” and/or “master mix” may refer to an aqueous solution comprising the various (some or all) reagents used to amplify a target nucleic acid. Such reactions may also be performed using solid supports or semi-solid supports (e.g., an array). The reactions may also be performed in single or multiplex format as desired by the user. These reactions typically include enzymes, aqueous buffers, salts, amplification primers, target nucleic acid, and nucleoside triphosphates. In some embodiments, the amplification reaction mix and/or master mix may include one or more of, for example, a buffer (e.g., Tris), one or more salts (e.g., MgC, KCl), glycerol, dNTPs (dA, dT, dG, dC, dU), recombinant BSA (bovine serum albumin), a dye (e.g., ROX passive reference dye), one or more detergents, polyethylene glycol (PEG), polyvinyl pyrrolidone (PVP), gelatin (e.g., fish or bovine source) and/or antifoam agent. Depending upon the context, the mixture can be either a complete or incomplete amplification reaction mixture. In some embodiments, the master mix does not include amplification primers prior to use in an amplification reaction. In some embodiments, the master mix does not include target nucleic acid prior to use in an amplification reaction. In some embodiments, an amplification master mix is mixed with a target nucleic acid sample prior to contact with amplification primers.

In some embodiments, the amplification reaction mixture comprises amplification primers and a master mix. In some embodiments, the amplification reaction mixture comprises amplification primers, a detectably labeled probe, and a master mix.

In some embodiments, the reaction mixture of amplification primers and master mix or amplification primers, probe and master mix are dried in a storage vessel or reaction vessel. In some embodiments, the reaction mixture of amplification primers and master mix or amplification primers, probe and master mix are lyophilized in a storage vessel or reaction vessel. In some embodiments, the disclosure generally relates to the amplification of multiple target-specific sequences from a single control nucleic acid molecule. For example, in some embodiments that single control nucleic acid molecule can include RNA and in other embodiments, that single control nucleic acid molecule can include DNA. In some embodiments, the target-specific primers and primer pairs are target-specific sequences that can amplify specific regions of a nucleic acid molecule, for example, a control nucleic acid molecule. In some embodiments, the target-specific primers can prime reverse transcription of RNA to generate target-specific cDNA. In some embodiments, the target-specific primers can amplify target DNA or cDNA. In some embodiments, the amount of DNA required for selective amplification can be from about 1 ng to 1 microgram. In some embodiments, the amount of DNA required for selective amplification of one or more target sequences can be about 1 ng, about 5 ng or about 10 ng. In some embodiments, the amount of DNA required for selective amplification of target sequence is about 10 ng to about 200 ng.

As used herein, the term “reaction vessel” generally refers to any container, chamber, device, or assembly, in which a reaction can occur in accordance with the present teachings. In some embodiments, a reaction vessel may be a microtube, for example, but not limited to, a 0.2 mL or a 0.5 mL reaction tube such as a Micro Amp™ Optical tube (Life Technologies Corp., Carlsbad, Calif.) or a micro-centrifuge tube, or other containers of the sort in common practice in molecular biology laboratories. In some embodiments, a reaction vessel comprises a well of a multi-well plate (such as a 48-, 96-, or 384-well microtiter plate), a spot on a glass slide, a well in a TaqMan™ Array Card or a channel or chamber of a microfluidics device, including without limitation a TaqMan™ Low Density Array, or a through-hole of a TaqMan™ OpenArray™ Real-Time PCR plate (Applied Biosystems, Thermo Fisher Scientific). For example, but not as a limitation, a plurality of reaction vessels can reside on the same support. An OpenArray™ Plate, for example, is a reaction plate 3072 through-holes. Each such through-hole in such a plate may contain a single TaqMan™ assay. In some embodiments, lab-on-a-chip-like devices available, for example, from Caliper or Fluidigm can provide reaction vessels. It will be recognized that a variety of reaction vessels are commercially available or can be designed for use in the context of the present teachings.

The terms “annealing” and “hybridizing”, including, without limitation, variations of the root words “hybridize” and “anneal”, are used interchangeably and mean the nucleotide base—pairing interaction of one nucleic acid with another nucleic acid that results in the formation of a duplex, triplex, or other higher-ordered structure. The primary interaction is typically nucleotide base specific, e.g., A:T, A:U, and G:C, by Watson-Crick and Hoogsteen-type hydrogen bonding. In certain embodiments, base-stacking and hydrophobic interactions may also contribute to duplex stability. Conditions under which primers and probes anneal to complementary sequences are well known in the art, e.g., as described in Nucleic Acid Hybridization, A Practical Approach, Hames and Higgins, eds., IRL Press, Washington, D.C. (1985) and Wetmur and Davidson, Mol. Biol. 31:349 (1968).

In general, whether such annealing takes place is influenced by, among other things, the length of the complementary portions of the complementary portions of the primers and their corresponding binding sites in the target flanking sequences and/or amplicons, or the corresponding complementary portions of a reporter probe and its binding site; the pH; the temperature; the presence of mono- and divalent cations; the proportion of G and C nucleotides in the hybridizing region; the viscosity of the medium; and the presence of denaturants. Such variables influence the time required for hybridization. Thus, the preferred annealing conditions will depend upon the particular application. Such conditions, however, can be routinely determined by persons of ordinary skill in the art, without undue experimentation. Preferably, annealing conditions are selected to allow the primers and/or probes to selectively hybridize with a complementary sequence in the corresponding target flanking sequence or amplicon, but not hybridize to any significant degree to different target nucleic acids or non-target sequences in the reaction composition at the second reaction temperature.

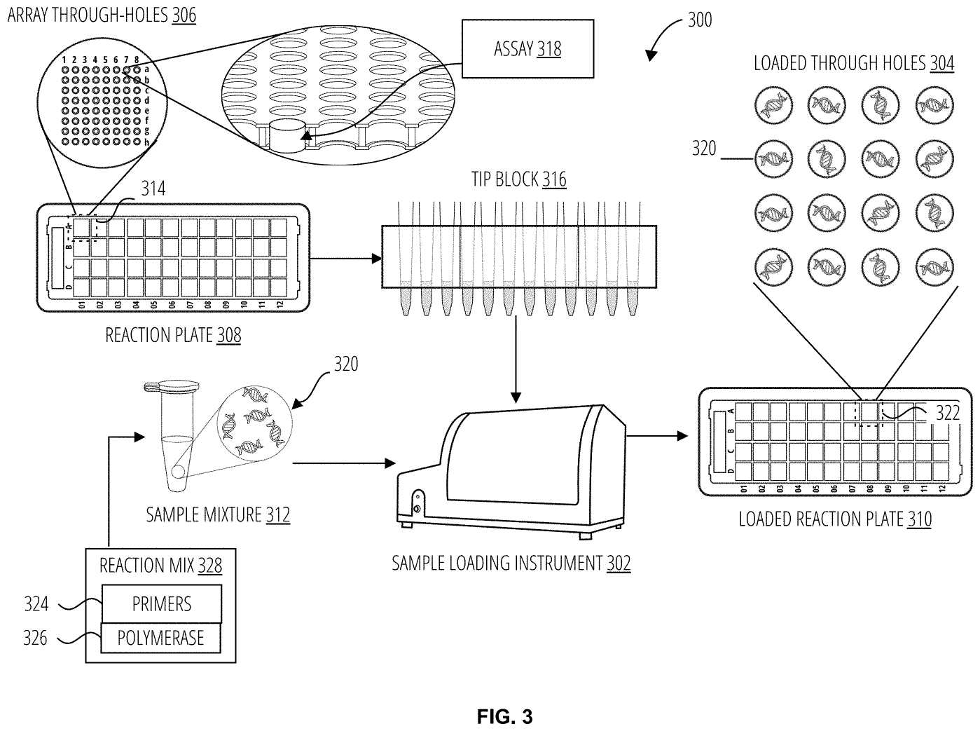

FIG. 3 illustrates plate preparation 300 for a reaction plate 308 before it is loaded into an qPCR instrument. A reaction plate 308 comprises a plurality of sub arrays, each sub array 314 comprising a plurality of through array through-holes 306. Each through-hole may serve as a reaction location for an assay 318. In some configurations, the reaction plate 308 comprises 48 subarrays with each sub array comprising 64 through-holes each capable of holding 33-nL of reaction volume. In the aforementioned configuration, the reaction plate 308 comprises 3072 through-holes.

Depending on the configuration of the reaction plate 308, some of the array through-holes 306 will include an assay 318 spotted within them. Each through-hole comprises a hydrophilic interior where the assay 318 may be spotted. The hydrophilic through-holes are also surrounded by hydrophobic surfaces that keep the reaction contained.

To accurately load a set volume into each desired array through-holes 306, a sample loading instrument 302 is utilized. The sample loading instrument 302 aliquots a set volume of a sample mixture 312 into each desired through-hole of the reaction plate 308. In some configurations, a tip block 316 is utilized by the sample loading instrument 302 to dispense the sample mixture 312 comprising the reaction mix 328 of primers 324 and a polymerase 326 into the through-holes of the reaction plate 308.

When the sample loading instrument 302 is operated, the tip block 316 may move across the reaction plate 308 allowing for a set volume of the sample mixture 312 to be delivered to the specific array through-holes 306. When the sample loading instrument 302 is completed its run, the reaction plate 308 is converted into a loaded reaction plate 310 where a plurality of sub arrays, for example sub array 322, comprises loaded through holes 304 comprising the target polynucleotide sequences 320.

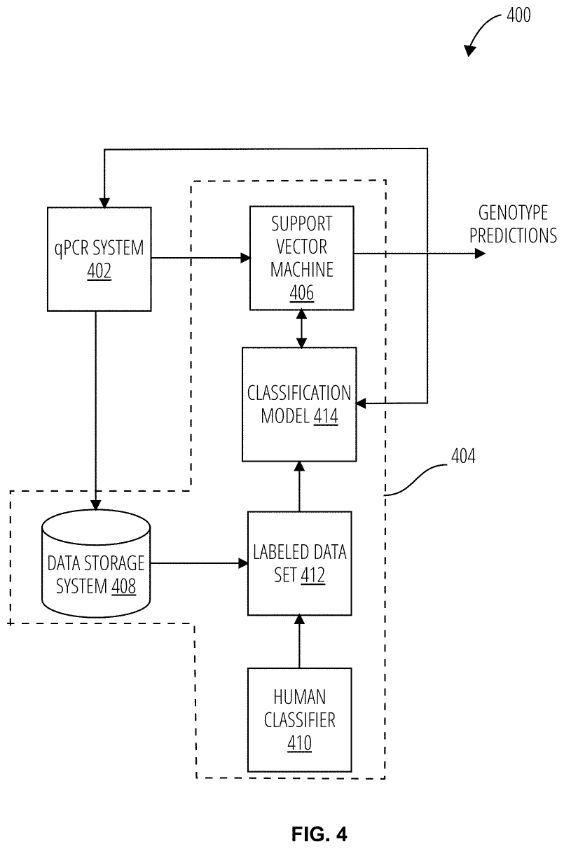

Referring to FIG. 4, a genotyping system 400 comprises a qPCR system 402 and a learning system 404. The learning system 404 further comprises a Support Vector Machine 406, a data storage system 408, a human classifier 410, a labeled data set 412, and a classification model 414.

The qPCR system 402 may be an embodiment of a qPCR system 200. The qPCR system 402 generates a signal comprising the intensity of FAM® and VIC® fluorescent dyes. This vector of intensities is then sent to the learning system 404, both the Support Vector Machine 406 and the data storage system 408. The vector may be further extended with values for number of centroid Minimum Cluster Separation Sigma (MCSS) clusters, assay address, MCSS values, etc.

The Support Vector Machine 406 receives the data vector from the qPCR system 402. The Support Vector Machine 406 may normalize the input raw data vector by utilizing min-max scaling or Z-score normalization. The Support Vector Machine 406 may then select a model from the classification model 414. The models may be selected from SVM linear, polynomial, and radial classifier (RBF) kernels. An RBF kernel may be as follows:

k(\vec{x}ᵢ, \vec{x}ⱼ)=exp(−γ∥\vec{x}ᵢ−\vec{x}ⱼ∥²) Equation 1

where x is the data vector and γ is a tunable parameter. The model may also have a hard-margin or a soft-margin. A soft-margin may be as follows:

min_{w,b}1/2∥w∥₂²+C Σₙ ζₙ s, t, yₙ (w^{T}xₙ+b)≥1−ζₙ Equation 2

where w and b are parameters for a hyperplane, xₙ is the data vector, yₙ is the ith target, ζ is a slack variable, and C is a tunable parameter. Each model may also have a set of hyperparameters. For example, a model utilizing a RBF kernel may have an associated γ value, such as a value between 10 and 1000. Additionally, a model utilizing a soft-margin may have an associated C value, such as a value between 0.01 and 30. The parameters may be selected to balance between operational efficiency and accuracy. The selected model may, for example, have a C value of 0.3 and a γ value of 300. The Support Vector Machine 406 utilizes the selected model to determine the genotype prediction of the data vector. As the dataset comprises three classes, a one-vs-the-rest (OvR) strategy is utilized to assign the genotype for new instances. This strategy utilizes one classifier per class (here, three classes). Each classifier then operates of the input data vector, for example, one classifier for the “11” state, one for the “12” state, and one for the “22” state. The Support Vector Machine 406 may select between the “11” state, the “12” state, and the “22” state based on the outputs of each classifier. The determined classification is then output.

The data storage system 408 stores data outputs from the qPCR system 402. The data storage system 408 may store the historical data utilize to train the models along with additional data generated by the qPCR system 402 after the models have been trained. New models may be generated from the update data sets stored in the data storage system 408. The data storage system 408 may further store data from more than one qPCR system 402.

The human classifier 410 applies labels to the data stored in the data storage system 408 to generate the labeled data set 412. The labels include the “11” state, the “12” state, and the “22” state. The labeled data set 412 is then utilized to train each classification model 414.

The classification model 414 may influence the operation of the qPCR system 402. The classification model 414 may utilize a different set of inputs than other classification model 414. The selected classification model 414 may then determine the output data vector from the qPCR system 402. Each classification model 414 may be trained by receiving a labeled data set 412, which may include Majority Genotype (MG) and Genotype Concordance (GC). MG is the genotype that has the highest frequency given a pair of assay-sample combination. As biologically a genotype of a qPCR reaction may be consistent, MG=max (G11, G12, G22), where, G11, G12, and G22 is genotype frequency for homozygotes (G11 and G22) and heterozygote (G12). GC is the percentage of number of instances of majority genotype in the historical data divided by the total number of qPCR reactions (assay-sample pair), GC=100*(MG instance/Total instances). The failed qPCR reactions are extracted from the stored data set, which consists of about half million instances (aka bad instances), then another half million instances that historically never failed (good instances) are randomly selected. This is input data utilized for training and testing. Each classification model 414 may include three classifiers. Each classifier determines a hyperplane (w and b values) to divide the labeled data set 412 into two categories—part of class or not part of the class. For example, a first classifier determines whether the data vector is “11” or not “11”. The second classifier determines whether the data vector is “12” or not “12”. The third classifier determines whether the data vector is “22” or not “22”. The accuracy between the existing (baseline) and the SVM-based genotyping are compared. The results for a model may be one of three categories: similar, better, and worse, in terms of statistical significance. The “best” prediction model is determined in terms of kernels and parameters of SVM after utilizing a grid search. Once a model is determined to be the “best”, its robustness is verified by four-fold cross validation. The input data set is divided into four groups. The model is then re-trained on three groups and tested with the four group. This is done four times, each group being the test group once. The training results show that an SVM-based algorithm predicts at least ˜20% higher accuracy than the conventional models based on the same data set. The results also show that SVM-RBF is able to rescue those 1 or 2-cluster data that the existing cannot make genotype predictions. In addition, the SVM-based algorithm rescues more than 50% of the uncalled and LowROX instances tagged by the conventional algorithm.

In some instances, the raw data comprises raw image data from the operation of the qPCR system. The raw image data comprises an array of pixel values generated by image sensor during the operation of the qPCR system.

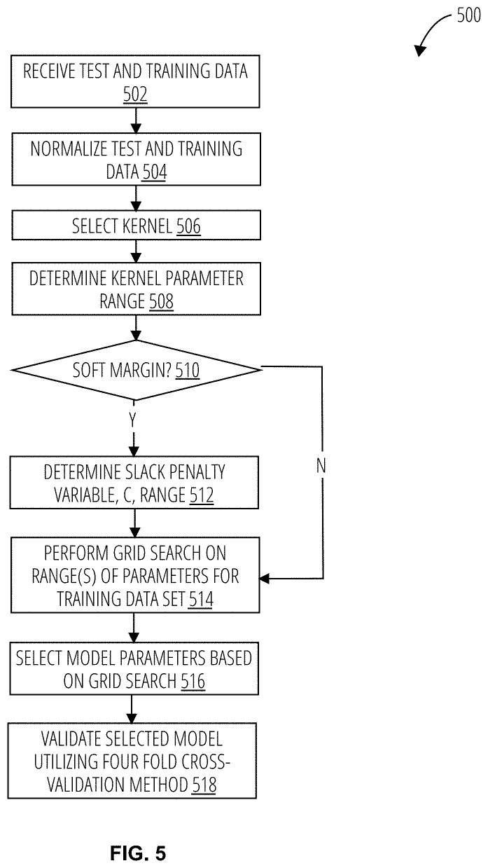

Referring to FIG. 5, a radial algorithm 500 receives test and training data (block 502). The test and training data is then normalized (block 504). Min-max scaling or Z-score normalization may be utilized. A specific kernel is selected (block 506). The kernel may include linear, polynomial, and radial classifier (RBF) kernels. The parameter range for the kernel is then determined (block 508). For example, for the RBF kernel a γ value may range between 10 and 1000. The radial algorithm 500 then determines whether the SVM is to have a hard-margin or a soft-margin (decision block 510). If the margin is soft, then the range for the slack penalty variable, C, is determined (block 512). For example, the range may be between 0.01 and 30. Once the range of C is determined or if the margin is hard, a grid search is performed on the range(s) of parameters for training data set (block 514). The grid search may transform the ranges of parameter into specific combination of parameters. For example, a grid value for the previously stated ranges may be C=0.01, γ=10; C=3, γ=10o; and C=30, γ=1000. Other values may be utilized. The test data may then be utilized to test the models generated by the grid search and select the model parameters (block 516). The model may be selected based on operation efficiency, accuracy, precision, etc. The selected model is then validated utilizing a fourfold cross-validation method (block 518). The test and training data may be divided into four groups. Three of the groups are utilized to re-train the model utilizing the selected parameters. The fourth group then is utilized to test the resulting model. This is performed four times, each group being a “test” group once. The model is evaluated on its ability to be trained on different datasets.

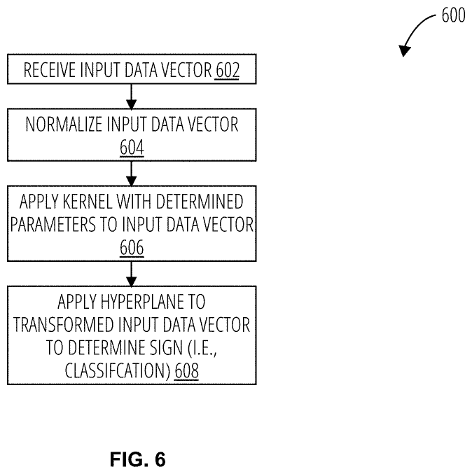

Referring to FIG. 6, an SVM qPCR assay model 600 receives input data vector (block 602). The input data vector may be an output from a qPCR system, which includes intensities for FAM® and VIC® fluorescent dyes, as well as other information in some embodiments, including values for number of centroid Minimum Cluster Separation Sigma (MCSS) clusters, assay address, MCSS values, etc. The input data vector is then normalized (block 604). A min-max scaling or Z-score normalization may be utilized. The SVM qPCR assay model 600 may determine the normalization method utilized to train the model and utilize the same method. A kernel with determined parameters is applied to the input data vector (block 606). This may transform the normalized input into the appropriate dimension space for the trained model. The hyperplane is applied to transformed input data vector to determine sign (i.e., classification) (block 608). As there are three or more classifications, multiple classifiers (hyperplanes) may be utilized. One hyperplane may be utilized per classification. Each hyperplane/classifier returning a sign, “+1” or “−1” indicating that the input data vector has that classification or does not have that classification, respectively. An input data vector with only one “+1” may be given that classification. For block 606 and block 608, the following may be utilized:

z→sgn(w·φ(z)−b)=sgn([Σᵢ₌₁ⁿ cᵢyᵢk(xᵢ, z)]−b) Equation 3

where φ is the kernel transform for the input data vector, and w and b are the parameters of the hyperplane for the model determined during training of the model. Here, there are three hyperplanes, as there are three classifications.

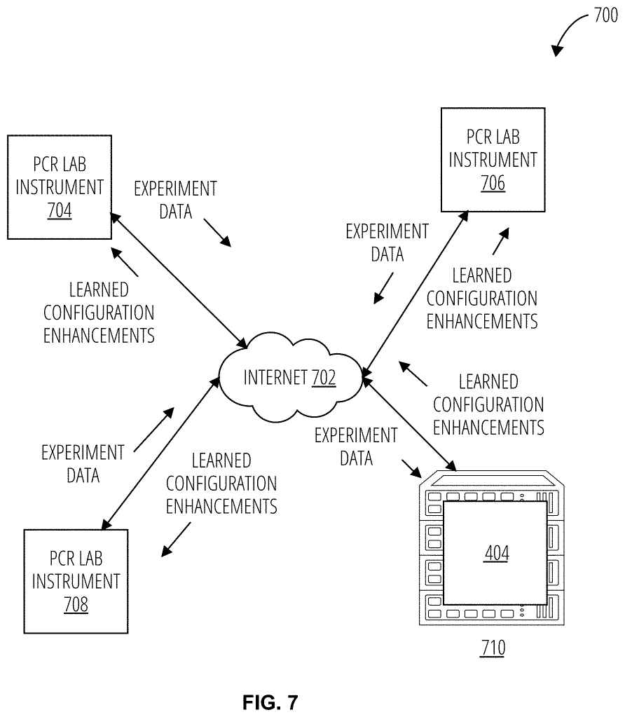

FIG. 7 illustrates a cloud learning and control system 700 in accordance with one embodiment. The cloud learning and control system 700 comprises a cloud analytic system 710 that comprises a learning system 404, for example one or more of the embodiments disclosed herein. The experimental data from a number of PCR runs or other experiments (e.g., PCR lab instrument 704, PCR lab instrument 706, and PCR lab instrument 708) are monitored over the Internet 702 or other network by the cloud analytic system 710. The cloud analytic system 710 processes the experimental data and provides learned configuration parameters as feedback to adjust the configuration settings PCR instruments for the current or future experiments.

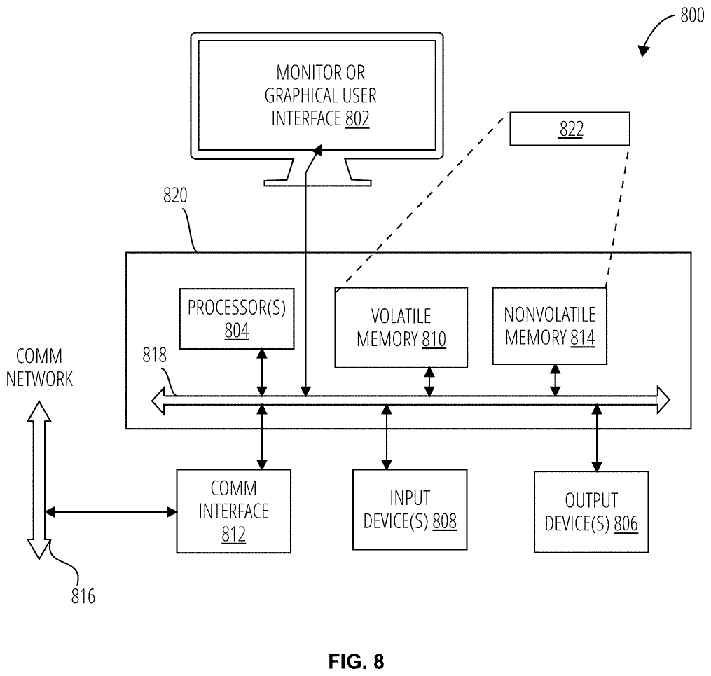

FIG. 8 is an example block diagram of a computing device 800 that may incorporate embodiments of the present invention. FIG. 8 is merely illustrative of a machine system to carry out aspects of the technical processes described herein, and does not limit the scope of the claims. One of ordinary skill in the art would recognize other variations, modifications, and alternatives. In one embodiment, the computing device 800 typically includes a monitor or graphical user interface 802, a data processing system 820, a communication network interface 812, input device(s) 808, output device(s) 806, and the like.

As depicted in FIG. 8, the data processing system 820 may include one or more processor(s) 804 that communicate with a number of peripheral devices via a bus subsystem 818. These peripheral devices may include input device(s) 808, output device(s) 806, communication network interface 812, and a storage subsystem, such as a volatile memory 810 and a nonvolatile memory 814.

The volatile memory 810 and/or the nonvolatile memory 814 may store computer-executable instructions and thus forming logic 822 that when applied to and executed by the processor(s) 804 implement embodiments of the analytical and control processes disclosed herein.

The input device(s) 808 include devices and mechanisms for inputting information to the data processing system 820. These may include a keyboard, a keypad, a touch screen incorporated into the monitor or graphical user interface 802, audio input devices such as voice recognition systems, microphones, and other types of input devices. In various embodiments, the input device(s) 808 may be embodied as a computer mouse, a trackball, a track pad, a joystick, wireless remote, drawing tablet, voice command system, eye tracking system, and the like. The input device(s) 808 typically allow a user to select objects, icons, control areas, text and the like that appear on the monitor or graphical user interface 802 via a command such as a click of a button or the like.

The output device(s) 806 include devices and mechanisms for outputting information from the data processing system 820. These may include the monitor or graphical user interface 802, speakers, printers, infrared LEDs, and so on as well understood in the art.

The communication network interface 812 provides an interface to communication networks (e.g., communication network 816) and devices external to the data processing system 820. The communication network interface 812 may serve as an interface for receiving data from and transmitting data to other systems. Embodiments of the communication network interface 812 may include an Ethernet interface, a modem (telephone, satellite, cable, ISDN), (asynchronous) digital subscriber line (DSL), FireWire, USB, a wireless communication interface such as Bluetooth or Wi-Fi, a near field communication wireless interface, a cellular interface, and the like.

The communication network interface 812 may be coupled to the communication network 816 via an antenna, a cable, or the like. In some embodiments, the communication network interface 812 may be physically integrated on a circuit board of the data processing system 820, or in some cases may be implemented in software or firmware, such as “soft modems”, or the like.

The computing device 800 may include logic that enables communications over a network using protocols such as HTTP, TCP/IP, RTP/RTSP, IPX, UDP and the like.

The volatile memory 810 and the nonvolatile memory 814 are examples of tangible media configured to store computer readable data and instructions to implement various embodiments of the processes described herein. Other types of tangible media include removable memory (e.g., pluggable USB memory devices, mobile device SIM cards), optical storage media such as CD-ROMS, DVDs, semiconductor memories such as flash memories, non-transitory read-only-memories (ROMS), battery-backed volatile memories, networked storage devices, and the like. The volatile memory 810 and the nonvolatile memory 814 may be configured to store the basic programming and data constructs that provide the functionality of the disclosed processes and other embodiments thereof that fall within the scope of the present invention.

Logic 822 that implements embodiments of the present invention may be embodied by the volatile memory 810 and/or the nonvolatile memory 814. Instructions of said logic 822 may be read from the volatile memory 810 and/or nonvolatile memory 814 and executed by the processor(s) 804. The volatile memory 810 and the nonvolatile memory 814 may also provide a repository for storing data used by the logic 822.

The volatile memory 810 and the nonvolatile memory 814 may include a number of memories including a main random-access memory (RAM) for storage of instructions and data during program execution and a read only memory (ROM) in which read-only non-transitory instructions are stored. The volatile memory 810 and the nonvolatile memory 814 may include a file storage subsystem providing persistent (non-volatile) storage for program and data files. The volatile memory 810 and the nonvolatile memory 814 may include removable storage systems, such as removable flash memory.

The bus subsystem 818 provides a mechanism for enabling the various components and subsystems of data processing system 820 communicate with each other as intended. Although the communication network interface 812 is depicted schematically as a single bus, some embodiments of the bus subsystem 818 may utilize multiple distinct busses.

It will be readily apparent to one of ordinary skill in the art that the computing device 800 may be a device such as a smartphone, a desktop computer, a laptop computer, a rack-mounted computer system, a computer server, or a tablet computer device. As commonly known in the art, the computing device 800 may be implemented as a collection of multiple networked computing devices. Further, the computing device 800 will typically include operating system logic (not illustrated) the types and nature of which are well known in the art.

Additional Terminology and Interpretation

Terms used herein should be accorded their ordinary meaning in the relevant arts, or the meaning indicated by their use in context, but if an express definition is provided, that meaning controls.

“Kernel” refers to kernel functions, which operate in a high-dimensional, implicit feature space without ever computing the coordinates of the data in that space, but rather by simply computing the inner products between the projections of all pairs of data in the feature space. This operation is often computationally cheaper than the explicit computation of the coordinates. When used with SVMs, this approach is called the “kernel trick”.

“Support Vector Machine” refers to supervised learning models with associated learning algorithms that analyze data used for classification and regression analysis. Given a set of training examples, each marked as belonging to one or the other of two categories, an SVM training algorithm builds a model that assigns new examples to one category or the other, making it a non-probabilistic binary linear classifier. An SVM model is a representation of the examples as points in space, mapped so that the examples of the separate categories are divided by a clear gap that is as wide as possible. New examples are then mapped into that same space and predicted to belong to a category based on which side of the gap they fall. In addition to performing linear classification, SVMs can efficiently perform a non-linear classification using what is called the kernel trick, implicitly mapping their inputs into high-dimensional feature spaces.

“Circuitry” herein refers to electrical circuitry having at least one discrete electrical circuit, electrical circuitry having at least one integrated circuit, electrical circuitry having at least one application specific integrated circuit, circuitry forming a general purpose computing device configured by a computer program (e.g., a general purpose computer configured by a computer program which at least partially carries out processes or devices described herein, or a microprocessor configured by a computer program which at least partially carries out processes or devices described herein), circuitry forming a memory device (e.g., forms of random access memory), or circuitry forming a communications device (e.g., a modem, communications switch, or optical-electrical equipment).

“Firmware” herein refers to software logic embodied as processor-executable instructions stored in read-only memories or media.

“Hardware” herein refers to logic embodied as analog or digital circuitry.

“Logic” herein refers to machine memory circuits, non-transitory machine readable media, and/or circuitry which by way of its material and/or material-energy configuration comprises control and/or procedural signals, and/or settings and values (such as resistance, impedance, capacitance, inductance, current/voltage ratings, etc.), that may be applied to influence the operation of a device. Magnetic media, electronic circuits, electrical and optical memory (both volatile and nonvolatile), and firmware are examples of logic. Logic specifically excludes pure signals or software per se (however does not exclude machine memories comprising software and thereby forming configurations of matter).

“Software” herein refers to logic implemented as processor-executable instructions in a machine memory (e.g. read/write volatile or nonvolatile memory or media).

Herein, references to “one embodiment” or “an embodiment” do not necessarily refer to the same embodiment, although they may. Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively, unless expressly limited to a single one or multiple ones. Additionally, the words “herein,” “above,” “below” and words of similar import, when used in this application, refer to this application as a whole and not to any particular portions of this application. When the claims use the word “or” in reference to a list of two or more items, that word covers all of the following interpretations of the word: any of the items in the list, all of the items in the list and any combination of the items in the list, unless expressly limited to one or the other. Any terms not expressly defined herein have their conventional meaning as commonly understood by those having skill in the relevant art(s).

Various logic functional operations described herein may be implemented in logic that is referred to using a noun or noun phrase reflecting said operation or function. For example, an association operation may be carried out by an “associator” or “correlator”. Likewise, switching may be carried out by a “switch”, selection by a “selector”, and so on.

1. A quality control system comprising:
a qPCR system comprising an assay;
a storage system coupled to receive first signals resulting from operation of the qPCR system on the assay; and
a computing system comprising logic to:
receive the first signals;
receive second signals comprising labeled data sets from the storage system;
operate a Support Vector Machine (SVM) to generate classifications for the first signals based on the second signals and to apply the classifications as operational feedback to the qPCR system.

2. The quality control system of claim 1, wherein the SVM comprises a radial basis function kernel.

3. The quality control system of claim 2, wherein the kernel comprises:
k(\vec{x}ᵢ, \vec{x}ⱼ)=exp(−γ∥\vec{x}ᵢ−\vec{x}ⱼ∥²).

4. The quality control system of claim 3, wherein the SVM further comprises a soft margin parameter of:
min_{w,b}1/2∥w∥₂²+C Σₙ ζₙ s, t, yₙ (w^{T}xₙ+b)≥1−ζₙ.

5. The quality control system of claim 1, wherein the storage system and SVM are provided by a cloud server system.

6. The quality control system of claim 1, wherein the classifications are applied as feedback to adapt the assay or use of the assay in the qPCR system.

7. The quality control system of claim 1, the SVM adapted to generate and adapt a model of the assay.

8. The quality control system of claim 7, wherein the model comprises one of SVM linear, polynomial, and radial classifier kernels.

9. The quality control system of claim 1, wherein the first signals and the second signals comprise raw image data from the operation of qPCR system.

10. A quality control method comprising:
operating a qPCR system on an assay to generate first signals;
receiving second signals comprising labeled data sets from a storage system;
operating a Support Vector Machine (SVM) to generate classifications for the first signals based on the second signals, wherein the SVM is adapted with a kernel comprising
k(\vec{x}ᵢ, \vec{x}ⱼ)=exp(−γ∥\vec{x}ᵢ−\vec{x}ⱼ∥²)
and a soft margin parameter comprising
min_{w,b}1/2∥w∥₂²+C Σₙ ζₙ s, t, yₙ (w^{T}xₙ+b)≥1−ζₙ; and
applying the classifications to adapt one or both of a process to generate the assay or operate the qPCR system.

11. The quality control system of claim 10, wherein the storage system and SVM are provided by a cloud server system.

12. The quality control system of claim 10, wherein the classifications are applied as feedback to adapt the manufacture of the assay or use of the assay in the qPCR system.

13. The quality control system of claim 10, the SVM adapted to generate and adapt a model of the assay.

14. The quality control system of claim 10, wherein the first signals and the second signals comprise raw image data from the operation of qPCR system.
